(19) United States
(12) Patent Application Publication
(10) Pub. No.: US 2014/0044744 A1
(43) Pub. Date: Feb. 13, 2014

(54) NOVEL HYPOALLERGENS

(71) Applicants: Kristiina Takkinen, Espoo (FI); Marja-Leena Laukkanen, Espoo (FI); Hans Söderlund, Espoo (FI); Sirpa Jylhä, Espoo (FI); Heidi Holkeri, Espoo (FI); Merja Niemi, Joensuu (FI); Juha Rouvinen, Joensuu (FI)

(72) Inventors: Kristiina Takkinen, Espoo (FI); Marja-Leena Laukkanen, Espoo (FI); Hans Söderlund, Espoo (FI); Sirpa Jylhä, Espoo (FI); Heidi Holkeri, Espoo (FI); Merja Niemi, Joensuu (FI); Juha Rouvinen, Joensuu (FI)

(73) Assignee: TEKNOLOGIAN TUTKIMUSKESKUS VTT, Espoo (FI)

(21) Appl. No.: 14/112,220

(22) PCT Filed: Apr. 18, 2012
(86) PCT No.: PCT/EP12/57047

§ 371 Date: Oct. 16, 2013

Related U.S. Application Data
(60) Provisional application No. 61/476,455, filed on Apr. 18, 2011.

(30) Foreign Application Priority Data
Apr. 18, 2011 (FI) 20115375

(51) Int. Cl. C07K 14/415

(52) U.S. Cl. CPC C07K 14/415
USPC 424/185.1; 530/370

(57) ABSTRACT

The present invention provides mutant polypeptides useful as hypoallergens. More specifically the present invention provides mutant Bet v 1 proteins and the use of such polypeptides as hypoallergens for desensitizing against birch pollen allergies. Furthermore, the invention provides vaccine formulations comprising such polypeptides; the use of such formulations; and to methods of vaccination against birch pollen allergy.

### FIELD OF THE INVENTION

The present invention relates to mutant polypeptides useful as hypoallergens. More specifically the present invention relates to mutant Bet v 1 proteins and the use of such polypeptides as hypoallergens for desensitizing against birch pollen allergies. Furthermore, the invention relates to vaccine formulations comprising such polypeptides; to the use of such formulations in vaccination; and to methods of vaccination against birch pollen allergy.

### BACKGROUND OF THE INVENTION

Allergies are caused by the immune reaction to commonly harmless proteins, allergens. Allergic diseases are reaching epidemic proportions all over the world. More than 25% of the population in industrialized countries suffer from type I allergy and the number is steadily increasing. Birch pollen allergy is a very common form of type I allergy. Bet v 1 is the major allergen of birch pollen. More information on the Bet v 1 allergen, its isoallergens and variants, is found on the WHO website www.allergen.org.

Type I allergy is based on the formation of immunoglobulin E (IgE) antibodies and the symptoms occur when an allergen molecule binds to two IgE antibodies bound to receptors on a mast cell or basophile surface and induces cross-linking of the IgE-FcεRI complexes. This triggers the degranulation of biological mediators, such as histamine and lipid mediators that cause inflammatory reactions and symptoms, such as allergic asthma, rhinitis, food and skin allergy, and even anaphylaxis.

The IgE is a large molecule that consists of two identical light and heavy chains. There are five domains in the heavy chain of IgE: VH, Cε1, Cε2, Cε3 and Cε4. The size of the complete IgE molecule is about 200 kDa. The crystal structures of the Cε2-Cε4 fragment bound to its FcεRI receptor, as well as the Cε2-Cε4 fragment have been determined (Garman et al., Nature 2000(406):259-266, and Wan et al., Nature Immunology, 2002(3):681-686).

In the last few years, the three-dimensional structures for a large number of different allergens have been determined. Structurally, these allergens vary considerably, and no common structural motif that could explain the capability of allergens to cause production of IgE antibodies has been identified. However, there are studies implicating that allergenicity is restricted to only a few protein families, thus raising evidence that structural features of proteins could also have a role in allergenicity (Rouvinen et al., PloS ONE 2010(5):e9037; Raudauer et al., J Allergy Clin Immunol. 2008(121):847-852).

A recent publication by Niemi et al., Structure 2007(15): 1413-21, discloses the crystal structure of an IgE Fab fragment in complex with β-lactoglobulin (BLG). It was also shown how two IgE/Fab molecules bind the dimeric BLG and that the IgE epitope was different when compared to known IgG epitope structures, being a “flat” surface located in the β sheet region.

Today the trend in the treatment of all allergic symptoms is towards an active induction of tolerance using allergen-specific desensitization instead of avoiding the allergen, which is often not possible, or merely treating the symptoms. Current desensitization therapy is based on allergens purified from natural sources, wherein batch to batch variations may lead to problems related to finding and maintaining the right dosage and efficiency of the treatment. These problems may lead to a potential risk of anaphylactic side effects and sensitization to new allergens.

The use of recombinant allergens for desensitizing would remove the disadvantages related to batch to batch variations, and the first recombinant allergens are in clinical trials (Valenta et al., Annu Rev Immunol 2010(28):211-41). The efficiency of such allergens in the clinic thus remains to be seen. Some modified recombinant allergens have been reported.

International patent publications WO 02/40676 and WO 03/096869 disclose numerous mutant forms of birch pollen allergen Bet v 1. These mutants were produced by introducing random mutations in the putative IgE binding site, based on sequence analysis of conserved surface structures of the Bet v1 polypeptide. WO 03/096869 discloses the use of four primary mutations on different “small groups” on the allergen surface.

International patent publication WO 2007/073907 discloses a Bet v 1 polypeptide comprising three amino acid substitutions or deletions at amino acid sites 54, 115 or 123. There is no evidence that these mutants have reduced histamine release capacity.

International patent publication WO 2009/024208 discloses a Bet v 1 mutant having at least four mutations in the area amino acids 100-125. However, due to the mutations the tree-dimensional structure of the polypeptide is lost, and there is no reported activity.

International patent publication WO 2008/092992 discloses a method of blocking the type I surface interaction of allergenic substances by modifying amino acid residues on non-continuous allergenic epitopes, i.e., on a planar surface with an area of 600-900 Å on the allergenic substance and suggests that hypoallergenic birch pollen proteins could be prepared accordingly.

There is a recognized and large need for safe and efficient vaccines and therapy products to meet the increasing medical problem of allergy. At present the market for safe and efficient therapies of allergy is underdeveloped.

### BRIEF DESCRIPTION [DISCLOSURE] OF THE INVENTION

The present invention relates to a recombinant birch pollen Bet v1 polypeptide based on a wild type amino acid sequence as depicted in SEQ ID NO: 4 or any other Bet v1 wild type isoform thereof, said polypeptide comprising at least one amino acid substitution at a position selected from the group consisting of amino acid residues 101, 137, 99, 80, 82, 84, 117, 119, 7, 9, 133, 141, and 145.

In a preferred embodiment, at least one amino acid substitution is at a position selected from the group consisting of amino acid residues E101, K137, S99, K80, N82, S84, S117, K119, T7, T9, V133, E141, and R145.

In a specific embodiment, a polypeptide according to the invention is represented by an amino acid sequence being selected from the group consisting of SEQ ID NO: 4-39.

In one embodiment the polypeptide according to this invention has an amino acid substitution located at amino acid position 101, 137 or 99, preferably E101, K137 or S99.

In one embodiment of the present invention, said substitution is S99 being replaced by tyrosine and in another embodiment said substitution is K137 being replaced by tyrosine. In a further embodiment, said substitution is E101 being replaced by lysine.

The hypoallergenic polypeptides according to the present invention have a histamine release capacity which is at least 20× reduced when compared to the histamine release capacity of the Bet v1 wild type. In one embodiment the polypeptide's histamine release capacity is reduced at least 100×.

The present invention also relates to a vaccine comprising at least one hypoallergenic polypeptide according to the present invention.

In one embodiment of the present invention said vaccine is for sublingual administration.

The present invention further relates to the use of a recombinant birch pollen Bet v1 polypeptide based on a wild type amino acid sequence as depicted in SEQ ID NO: 4 or any Bet v1 wild type variant thereof, said polypeptide comprising at least one amino acid substitution at a position selected from the group consisting of amino acid residues E101, K137, S99, K80, N82, S84, S117, K119, T7, T9, V133, E141, and R145 as a vaccine, alone or in combination with at least one pharmaceutically acceptable adjuvant.

The present invention also relates to a method of vaccinating against birch pollen allergy, said method comprising administering to a subject in need thereof a composition comprising at least one of the hypoallergenic polypeptide according to the present invention and at least one pharmaceutically acceptable adjuvant.

The present invention also relates to a recombinant birch pollen Bet v 1 polypeptide based on a wild type amino acid sequence as depicted in SEQ ID NO: 4 or any other Bet v1 wild type isoform, said polypeptide comprising at least one amino acid substitution at a position selected from the group consisting of amino acid residues 101, 137, 99, 80, 82, 84, 117, 119, 7, 9, 133, 141, and 145 for use as a vaccine.

### BRIEF DESCRIPTION OF THE FIGURES

In the following the invention will be described in greater detail by means of preferred embodiments with reference to the attached drawings, in which

FIG. 1 illustrates a first putative epitope of Bet v1 derived in Example 1;

FIG. 2 illustrates a second putative epitope of Bet v1 derived in Example 1;

FIG. 3 is an amino acid sequence alignment of 36 isoforms of Bet v 1;

FIG. 4 is a schematic presentation of the bacterial expression units for production of recombinant allergens, wherein Ptac is a promoter, PelB SS is the signal sequence linked to the coding region of recombinant allergens and the stars illustrate the amino acid substitution sites;

FIG. 5 shows the nucleic acid sequences (SEQ ID NOs 1-3) of the Bet v1 wild type polypeptide (A), the S99Y polypeptide (B), the K137 polypeptide (C) and the E101K polypeptide used in Example 2;

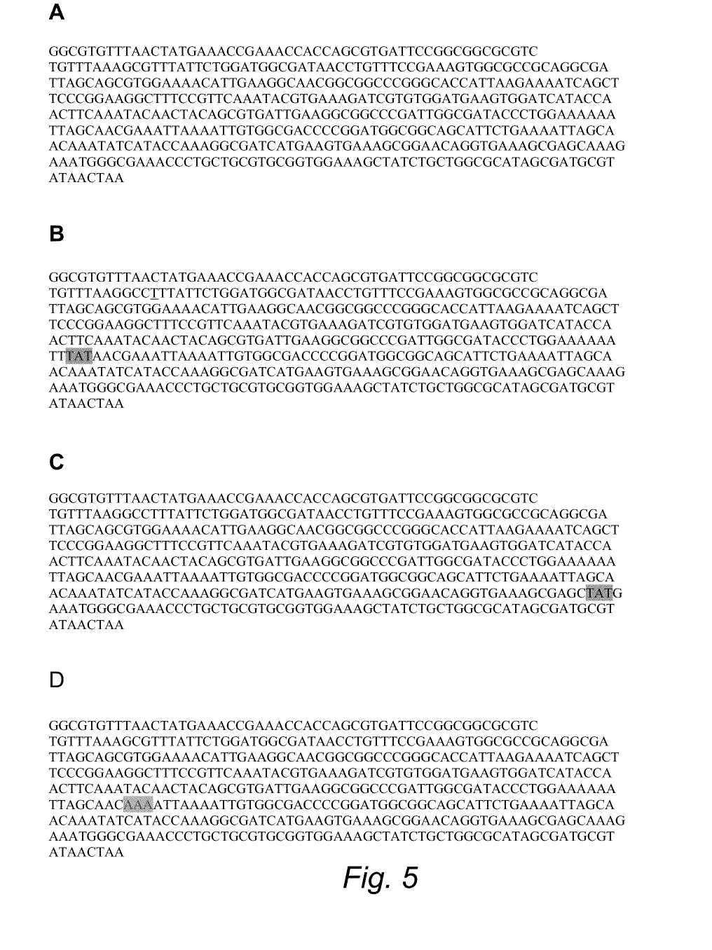

FIG. 6 shows the competitive inhibition of serum IgE binding to Bet v 1 with recombinant Bet v 1, Bet v 1 S99Y and E101K polypeptides;

FIG. 7 shows the results of histamine release experiments with recombinant Bet v 1, Bet v1 S99Y and K137Y polypeptides; and

FIG. 8 shows the native ESI FT-ICR mass spectra of the recombinant Bet v 1 wild type and recombinant Bet v 1 mutant E101K at a concentration of 3 μM.

### DETAILED DESCRIPTION OF THE INVENTION

In the following description, examples and claims both three-letter and one-letter codes are used for amino acids. To denominate amino acid sites in the polypeptides according to the present invention, the following codes are used: S99 means that there is a serine at position 99, whereas S99Y, means that the serine at position 99 has been replaced by tyrosine.

Birch pollen allergy is a very common form of allergy and pollen of the white birch (Betula verrucosa) is one of the main causes of Type I allergy reactions in Europe and North America. It is estimated that about 10-15% of the population may suffer from birch pollen allergy. Furthermore, other allergens, such as apple allergens, cross-react with birch pollen specific IgE causing allergic reactions even when the subject is not subjected to pollen.

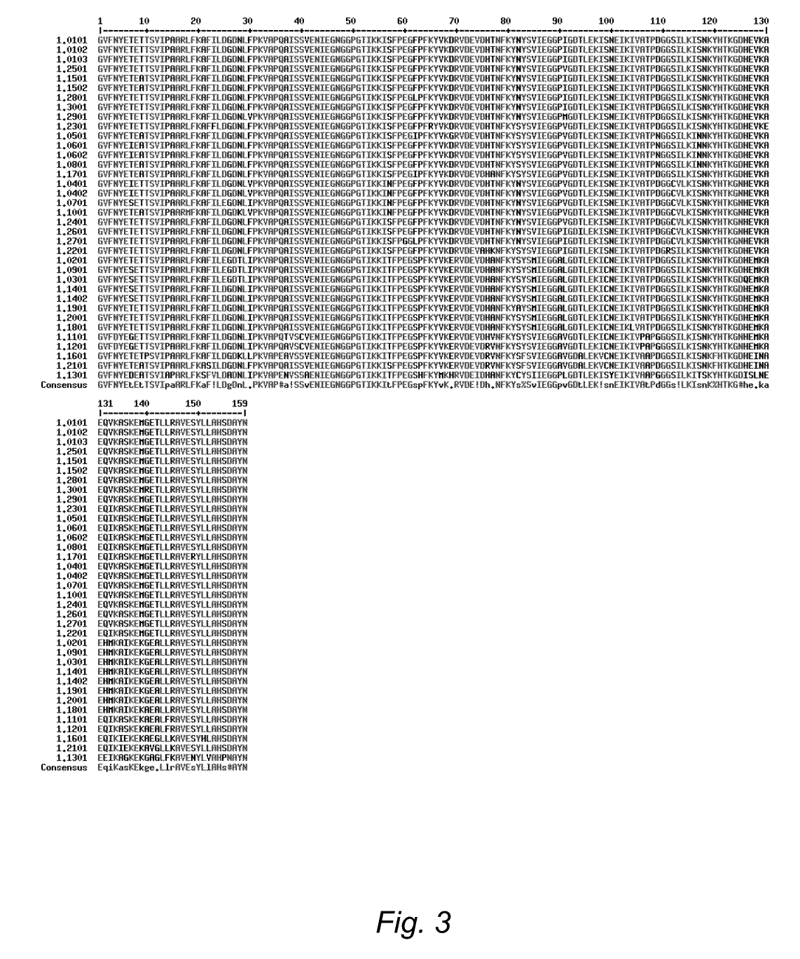

Bet v 1 is the major allergen of birch pollen and it is responsible for the IgE binding in more than 95% of birch pollen allergic subjects. Bet v1 is a protein having a molecular weight of 17 kD. The amino acid sequence of wild type Bet v1 is given in SEQ ID NO: 4. The WHO allergen website (www.allergen.org) lists thirty-six (36) isoforms of Bet v 1, which have been sequence aligned in FIG. 3. The alignment shows that Bet v 1 is highly conserved. The isoform used as a wild-type Bet v1 in the present invention is isoform Bet v1a (Bet v 1.0101), but any one of these isoallergens may be used to provide a hypoallergenic variant according to the present invention. The wild-type sequence (SEQ ID NO: 4) is intended to include all Bet v 1 isoform sequences.

The amino acid sequences of all 36 Bet v1 isoforms are disclosed in the sequence listing, as follows: 1.0101 (SEQ ID NO: 4), 1.0102 (SEQ ID NO: 5), 1.0103 (SEQ ID NO: 6), 1.2501 (SEQ ID NO: 7), 1.1501 (SEQ ID NO: 8), 1.1502 (SEQ ID NO: 9), 1.2801 (SEQ ID NO: 10), 1.3001 (SEQ ID NO: 11), 1.2901 (SEQ ID NO: 12), 1.2301 (SEQ ID NO: 13), 1.0501 (SEQ ID NO: 14), 1.0601 (SEQ ID NO: 15), 1.0602 (SEQ ID NO: 16), 1.0801 (SEQ ID NO: 17), 1.1701 (SEQ ID NO: 18), 1.0401 (SEQ ID NO: 19), 1.0402 0801 (SEQ ID NO: 20), 1.0701 0801 (SEQ ID NO: 21), 1.1001 0801 (SEQ ID NO: 22), 1.2401 0801 (SEQ ID NO: 23), 1.2601 0801 (SEQ ID NO: 24), 1.2701 0801 (SEQ ID NO: 25), 1.2201 0801 (SEQ ID NO: 26), 1.0201 0801 (SEQ ID NO: 27), 1.0901 0801 (SEQ ID NO: 28), 1.0301 0801 (SEQ ID NO: 29), 1.1401 0801 (SEQ ID NO: 30), 1.1402 0801 (SEQ ID NO: 31), 1.1901 0801 (SEQ ID NO: 32), 1.2001 0801 (SEQ ID NO: 33), 1.1801 0801 (SEQ ID NO: 34), 1.1101 0801 (SEQ ID NO: 35), 1.1201 0801 (SEQ ID NO: 36), 1.1601 0801 (SEQ ID NO: 37), 1.2101 0801 (SEQ ID NO: 38), and 1.1301 0801 (SEQ ID NO: 39), respectively.

The isoforms of Bet v 1 include variants which have different allergenic potential. The isoforms of Bet v 1 are at least 94% identical to Bet v 1 wild type amino acid sequence of SEQ ID NO: 4. For instance, isoforms Bet v 1.0401 with 96% amino acid residue identity and Bet v 1.1001 with 94% residue identity to Bet v 1.0101 have been identified as natural hypoallergens, because they were poor inducers of a mediator release.

The present invention provides mutated hypoallergenic variants of Bet v 1, which are useful as vaccines for immunizing subjects in need thereof and thus preventing and/or alleviating allergy and desensitizing subjects suffering from allergy against birch pollen. The recombinant birch pollen Bet v1 polypeptides according to the present invention, have a wild type amino acid sequence mutated as to include at least one amino acid substitution at a position selected from the group consisting of amino acid residues E101, K137, S99, K80, N82, S84, S117, K119, T7, T9, V133, E141, and R145. The polypeptides according to the present invention are hypoallergenic, and exhibit a histamine release capacity which is at least 20×, preferably 100×, reduced when compared to the histamine release capacity of the unmutated Bet v1 wild type.

The hypoallergenic polypeptides according to the present invention are useful as vaccines against allergy, especially birch pollen allergy. Vaccines comprising polypeptides according to the present invention are formulated according to standard pharmaceutical procedures known to skilled persons in the art. Vaccines according to the present invention are especially suited for sublingual administration. Preferably, the vaccine composition of the present invention comprises at least one recombinant hypoallergenic Bet v 1 polypeptide of the invention and at least one pharmaceutically acceptable diluent or adjuvant, such as saline, buffer, aluminum hydroxide and like.

Hypoallergenic variants according to the present invention are obtained by mutating some (1-5) specific amino acid residues, e.g. residues with bulky side chains, located on the epitope surface of Bet v 1. The selected amino acid residues are those, whose side chains point outside towards the solvent. Mutating such residues cause minimal change to the basic 3-dimensional structure of the allergen. Preferably, however, the mutagenesis modifies the surface of the epitope to such an extent that the binding and cross-linking of IgE antibodies on the mast cell surface is prevented or strongly reduced, while the over-all structure of the variant is still very similar to that of the wild type allergen. Such a mutation favours the induction of IgG and other protective antibodies, having the ability of binding both to the wild-type allergen and to the mutated variant allergen. The effect of the mutation is determined as a lower affinity of the allergen specific IgE antibody towards the modified Bet v 1 allergen. Preferably the mutation decreases the affinity of the specific IgE antibody at least tenfold, preferably at least 20-fold, and more preferably 20- to 100-fold, and most preferably more than 100-fold. The resulting modified Bet v 1 allergen can be used to evoke tolerance against birch pollen in allergic patients.

The hypoallergenic variant polypeptides according to the present invention, useful in allergen-specific desensitization, possess two features: 1) the ability to strongly reduce an IgE-mediated reaction; and 2) a retained wild-type 3D folding, and thus the capability of inducing the production of IgG-antibodies capable to bind wt allergen.

The knowledge of the structure of the IgE binding epitope would greatly simplify the design of hypoallergenic variants. However, the structure of Bet v 1 complexed with IgE antibody is unfortunately not available. The use of peptides in the epitope scanning is also unreliable and actually useful only when scanning linear epitopes (Niemi et al., Structure 2007(15): 1413-21). The conformation as well as the physical properties, e.g., solubility, of a single peptide may differ markedly from those of corresponding portion of a polypeptide chain forming part of a native protein structure. Therefore, the design of the mutant Bet v 1 allergens was based on molecular surface analysis using molecular graphics programmes, such as PyMOL, to elucidate the structure of the epitope and to test potential hits by preparing and testing the mutants.

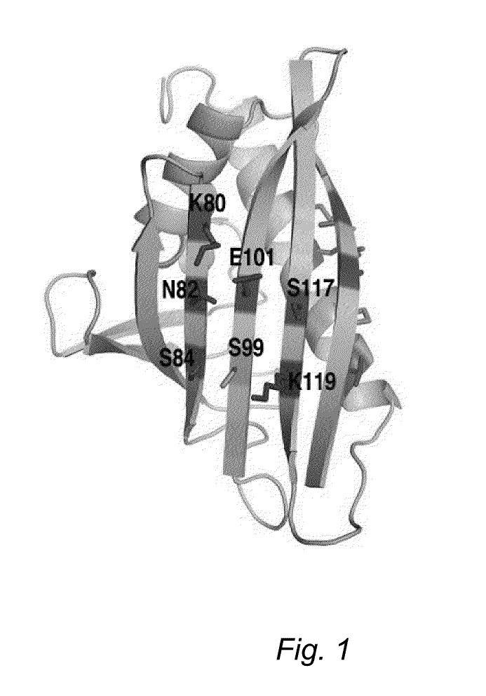

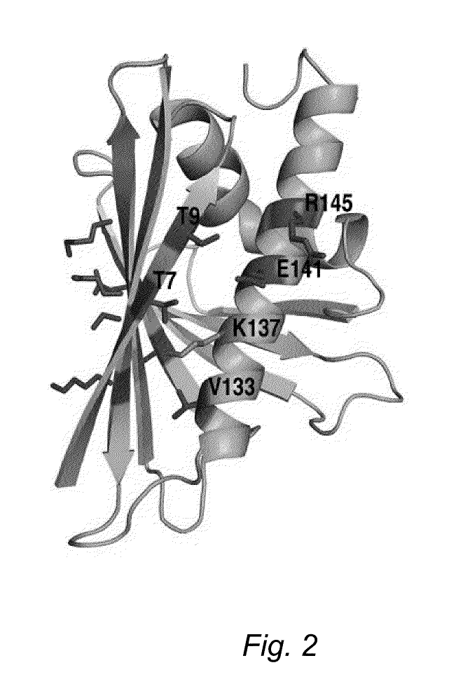

The crystal structure of Bet v 1 (protein data bank code 1BV1) was used to define the quaternary structure of Bet v 1. The PDBePISA internet server was used for creating coordinates for the symmetric dimer of Bet v 1. It has been estimated that the distance between IgE antibodies in the cluster on the mast cell surface is about 5 nm (Knol, E F; Mol. Nutr. Res. 50 (2006):620). By studying the molecular surface of the Bet v 1 dimer around the two-fold symmetry axis within a distance of 2.5 nm from the symmetry axis, two putative epitopes (FIG. 1 and FIG. 2) were identified on the molecular surface of Bet v 1.

The putative epitope 1 is composed of amino acid residues V2-E6; R70-D75; N78-S84; E96-K103; and K115-H121, whereas putative epitope 2 is composed of amino acid residues F3-V12; A130-L152; and T107-D109.

These putative epitopes were carefully analyzed, in order to identify amino acid residues, which could serve as mutation points. Preferred mutations points should have the ability to decrease the binding of the allergen to IgE antibodies but still maintain the three-dimensional structure of the wild-type allergen. The first putative epitope includes amino acid residues K80, N82, S84, S99, E101, S117, and K119. The second putative epitope includes residues T7, T9, V133, K137, E141, and R145. Residues S99, E101K and K137 were considered as the most interesting mutation points, as they are located in the center of the two putative epitopes.

These three residues are highly conserved in all 36 isoforms of Bet v1, the only variation being residue 99, which exists as either serine (in 24 isoforms) or cystein (in 12 isoforms). Residue 80, 84, 119, 141 and 145 are conserved, whereas residues 82, 117, 7, 9, 133 vary slightly, as shown in FIG. 3.

The next step was to select an appropriate mutation for each residue. As an example, S99 (in epitope 1) is a small hydrophilic and neutral amino acid residue. The mutation which would interfere IgE binding would thus be of “opposite” nature, i.e., large and/or charged, for example Phe, Asp, Glu, Lys, Arg, Tyr, His, or Trp. Similarly, E101 in epitope 1, can be replaced by a residue with the opposite charge (Lys, Arg) or with a hydrophobic residue (Tyr, Trp, Phe, Val, Ile, or Leu). As a second example, the side chain of K137 (in the epitope 2) is long, flexible, and positively charged. The mutation to smaller residue would not probably be helpful because it would not prevent binding to the antibody because of free space. K137 would thus be substituted by negatively charged residue, Asp, Glu, or by a rigid large residue such as Trp, Tyr, Phe, Ile, or Met.

Substitutions at residues K80, N82, S84, E101, S117, and K119 and residues T7, T9, V133, E141, and R145, respectively, could be designed correspondingly. Table 1 lists potential substitutions, which would yield Bet v1 hypoallergenic mutants according to the present invention.

TABLE 1

| | | | | | |
|---|---|---|---|---|---|
| Bet v 1 wt | mutant 1 | mutant 2 | mutant 3 | mutant 4 | mutant 5 |

Putative epitope 1

| | | | | | |
|---|---|---|---|---|---|
| S99 small | K large charged | R large charged | D charged | Y large | V large |
| hydrophilic | | | | hydrophobic | hydrophobic |
| E101 charged | K large | R large | Y large | I large | W large |
| | oppositely charged | oppositely charged | hydrophobic | hydrophobic | hydrophobic |
| K80 charged | Y large hydrophobic | E oppositely | W large | I large | L large |
| | | charged | hydrophobic | hydrophobic | hydrophobic |
| S84 small | K large charged | R large charged | D charged | E charged | Y large |
| hydrophilic | | | | | hydrophobic |
| N82 hydrophilic | K large charged | R large charged | Y large | E charged | L hydrophobic |
| | | | hydrophobic | | |
| S117 small | K large charged | R large charged | D charged | Y large | L large |
| hydrophilic | | | | hydrophobic | hydrophobic |
| K119 charged | E oppositely | Y hydrophobic | L hydrophobic | W hydrophobic | I hydrophobic |
| | charged | | | | |

Putative epitope 2

| | | | | | |
|---|---|---|---|---|---|
| T7 small | E charged | Y large | K large charged | R large charged | W large |
| hydrophilic | | hydrophobic | | | hydrophobic |
| T9 small | E charged | Y large | K large charged | R large charged | W large |
| hydrophilic | | hydrophobic | | | hydrophobic |
| V133 hydrophobic | Y large | E charged | W large | K charged | R charged |
| | hydrophobic | | hydrophobic | | |
| K137 charged | E oppositely | Y hydrophobic | L hydrophobic | W hydrophobic | I hydrophobic |
| | charged | | | | |
| E141 charged | K oppositely | R oppositely | Y large | W large | I large |
| | charged | charged | hydrophobic | hydrophobic | hydrophobic |
| R145 charged | Y hydrophobic | E oppositely | W hydrophobic | I hydrophobic | L hydrophobic |
| | | charged | | | |

In a preferred embodiment of the invention, the polypeptide of the invention comprises an amino acid sequence selected from the group consisting of SEQ ID NO: 41-53, or isoform thereof. Preferably, the polypeptide of the invention has an amino acid sequence selected from the group consisting of SEQ ID NO: 41-53, or isoform thereof.

In a further preferred embodiment, the polypeptide comprises an amino acid sequence selected from the group consisting of SEQ ID NO: 41, wherein amino acid 101 is K, SEQ ID NO: 42, wherein amino acid 137 is K, SEQ ID NO: 43, wherein amino acid 99 is K, SEQ ID NO: 44, wherein amino acid 80 is Y, SEQ ID NO: 45, wherein amino acid 82 is K, SEQ ID NO: 46, wherein amino acid 84 is K, SEQ ID NO: 47, wherein amino acid 117 is K, SEQ ID NO: 48, wherein amino acid 119 is E, SEQ ID NO: 49, wherein amino acid 7 is E, SEQ ID NO: 50, wherein amino acid 9 is E, SEQ ID NO: 51, wherein amino acid 133 is Y, SEQ ID NO: 52, wherein amino acid 141 is K, and SEQ ID NO: 53, wherein amino acid 145 is Y, or isoform thereof. Preferably, the polypeptide has anyone of these amino acid sequences SEQ ID NO: 41-53 with abovementioned substitution, respectively, or isoform thereof. SEQ ID NO: 41 discloses Bet v 1 polypeptide sequence, wherein amino acid at position 101 is a substitution and not wild type amino acid E. Similarly SEQ ID NOs: 42-53 show Bet v 1 polypeptides, wherein amino acids at positions 137, 99, 80, 82, 84, 117, 119, 7, 9, 133, 141, and 145, respectively, are substitutions and not wild type amino acids.

In a further preferred embodiment of the invention, the substitutions of Bet v 1 are at least at positions E101 and K137, E101 and S99, E101 and K80, E101 and N82, E101 and S84, E101 and S117, E101 and K119, E101 and T7, E101 and T9, E101 and V133, E101 and E141, E101 and R145, K137 and S99, K137 and K80, K137 and N82, K137 and S84, K137 and S117, K137 and K119, K137 and T7, K137 and T9, K137 and V133, K137 and E141, K137 and R145, S99 and K80, S99 and N82, S99 and S84, S99 and S117, S99 and K119, S99 and T7, S99 and T9, S99 and V133, S99 and E141, S99 and R145.

In one preferred embodiment of the invention, there are altogether at least two, three or four amino acid substitutions. Preferably, the polypeptide of the invention has two, three, four, five, six, seven, eight, nine or ten substitutions. More preferably, the polypeptide has two, three or four substitutions. One of the advantages of the invention is that only a small number of substitutions (at least two) are needed for the desired effects.

The modified Bet v1 hypoallergens according to the present invention are useful as vaccines. Conventional allergy vaccination is typically carried out as multiple subcutaneous immunizations over an extended time period, e.g., one to two years. In order to minimize the risk of anaphylactic reactions, the immunization scheme is applied in two phases, an initial up-dosing phase and a maintenance phase. The up-dosing phase starts with minute doses, which are then slowly increased, typically over a 16-week period until the maintenance dose is reached. The maintenance phase typically comprises injections every sixth week. Such a vaccination regime is tedious for the patient, requiring a long-term commitment. Moreover, it puts high impact on the steady quality of the vaccine, in terms of safety and reproducibility. The patients need to be strictly monitored, often hospitalized, after each injection.

As the histamine release capacity of the hypoallergens according to the present invention is substantially reduced, the dosing-up phase could be significantly shorter than that of a conventional allergy vaccination, or at best no dosing-up scheme could be needed. Modified, recombinant hypoallergens according to the present invention do not present any batch-to-batch variation. Thus, close monitoring of the dose-response and possible side-reactions is not needed.

The present invention further relates to a method of vaccinating against birch pollen allergy, said method comprising administering to a subject in need thereof a composition comprising at least one hypoallergenic polypeptide of the invention and at least one pharmaceutically acceptable adjuvant. Such a vaccination schedule and amount of hypoallergic polypeptide are used, which are effective for inducing the production of protective antibodies against birch pollen.

A “subject” of vaccination is a human (adult, child or adolescent) or an animal. Preferably, the animal is any domestic animal such as a dog, cat, horse, cow, sheep or pig. A “subject in need thereof” means a human or an animal suffering from birch pollen allergy.

For instance, a hypoallergen according to the present invention is formulated as conventional vaccine formulations, such as aluminium hydroxide-adsorbed vaccines, using methods well known in the art (Niederberger et al., PNAS, 101(2):14677-82, 2004). However, the hypoallergens according to the present invention may be administered by other suitable vaccination routes and schemes, such as oromucosal or sublingual administration, using methods and formulations known in the art. See, e.g., European Patent publication EP 1812059.

The modified Bet v1 hypoallergens could be used in concentrations of e.g., 0.5 μg/ml, 5 μg/ml or 50 μg/ml. Exemplary doses may vary between 0.05 μg and 2 μg during a possible dosing-up phase, and between 3-15 μg during the maintenance phase, preferably 5-15 μg, most preferably about 10 μg, depending on the severity of the allergy, the age and medical history of the patient. A suitable dose is easily decided by a clinician familiar with treating and preventing allergy.

International patent publication WO 04/047794 discloses a solid fast-dispersing dosage form for sublingual administration of an allergy vaccine, and US patent application 2009/0297564 discloses a liquid vaccine formulation for oromucosal administration.

The modified Bet v 1 hypoallergens according to the present invention are also suitable for sublingual administration using sublingual drops. For this purpose the hypoallergenic polypeptides are provided in saline. A safe and effective dose range for administration of the polypeptides, as well as the dosing regimen capable of eliciting a desired immune response is determined during clinical development of the vaccine candidates according to the present invention, using methods and schemes known in the art.

A maximum tolerated single dose of a hypoallergen according to the present invention is determined in a study in allergic male and female subjects, which are exposed to increasing sublingual doses. When the maximal tolerated dose of predefined dose is reached, the study is adapted to a dose ranging study with daily dosing, where the dose levels differ by a factor of 2 to 4. The initial dose is in the range of 10-100 μg, and the study provides the maximal tolerated sublingual dose, which may be as high as 20 mg.

Thereafter dose escalation and dose ranging over a wide dose range administered daily or weekly are studied. The safety of the vaccination dose range is preliminary tested with a Skin Prick Test prior to administering multiple doses. These studies provide primarily immunological parameters, and secondarily, eventual efficacy after challenge by birch pollen.

The hypoallergenic polypeptide vaccines according to the present invention should elicit a T-cell response detectable as a shift from TH2- to TH1-type. Production of IgG antibodies should be demonstrable before entering allergenic challenge testing.

Finally, a study in allergic patients is performed, as a double blind, randomized placebo controlled desensitization study in allergic male and female subjects exposed to a number of sublingual doses during 3-6 months, with a follow up for 12 months initially. The subjects will be challenged by allergen prior to the start of the study as well as every six months thereafter in a double blind manner.

The study will show a statistically a clinically significant difference between the groups receiving placebo and a hypoallergen vaccine according to the present invention, when they are challenged to the native allergen.

### EXAMPLES

The following examples are given to further illustrate embodiments of the present invention, but are not intended to limit the scope of the invention. It will be obvious to a person skilled in the art, as technology advances, that the inventive concept can be implemented in various ways. The invention and its embodiments are thus not limited to the examples described herein, but may vary within the scope of the claims.

### Example 1

### Design of the Bet v 1 Mutations

The goal in the hypoallergen design is to achieve a mutant allergen whose ability to bind and cross-link IgE-antibodies on the mast-cell surface is strongly reduced but which still maintains a very similar structure as the wild type allergen. This would favour the induction of IgG and other antibodies which would have ability to bind both to wild-type allergen and mutant allergen.

The knowledge of IgE epitope would greatly simplify design. However, there is no structure of Bet v 1 complexed with IgE antibody available. The use of peptides in the epitope scanning is also unreliable (Niemi et al., 2007). The only method to suggest an epitope is to study the molecular surface of Bet v 1 allergen and test the possible hit by preparing mutants. Firstly, we identified to putative epitopes (FIG. 1 and FIG. 2) on the molecular surface of Bet v 1. Secondly, we selected such residues on these putative epitopes which mutated would maintain a three-dimensional structure similar to the wild-type allergen and still have the ability to decrease binding to IgE antibodies. The first putative epitope includes amino acid residues K80, N82, S84, S99, E101, S117, and K119. The second putative epitope includes residues T7, T9, V133, K137, E141, and R145.

The third step is to select mutation for each residue. As an example, S99 (in the epitope 1) is a small hydrophilic and neutral residue. The mutation which would interfere IgE binding would thus be “opposite”, large and/or charged, for example Phe, Asp, Glu, Lys, Arg, Tyr, His, Trp. In the case of E101K in epitope 1, mutations interfering with the IgE binding could include using residues with an opposite charge (Lys, Arg) or using hydrophobic residues (Tyr, Trp, Phe, Val, Ile, Leu). As a second example, the side chain of K137 (in the epitope 2) is long, flexible, and positively charged. The mutation to smaller residue would not probably be helpful because it would not prevent binding to the antibody because of free space. K137 would thus be substituted by negatively charged residue, Asp, Glu, or by a rigid large residue such as Trp, Tyr, Phe, Ile, and Met.

### Example 2

### Cloning of the Recombinant Bet v 1 Molecules

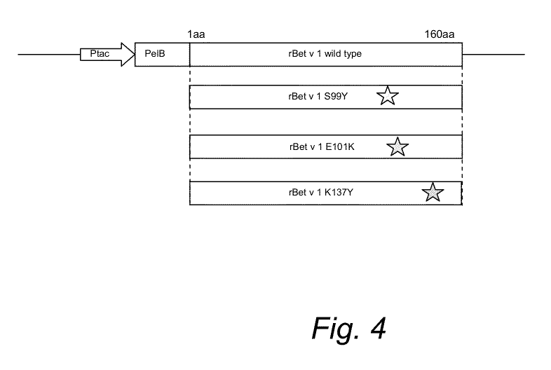

To produce the wild type and the mutants of the recombinant Bet v 1 molecules (rBet v 1) the cDNAs encoding these particular proteins were cloned into a bacterial expression plasmid (FIG. 4). First, the rBet v 1 cDNAs designed in Example 1, with the codon optimization for Escherichia coli production in vector pUC57 (wt, S99Y, E101K and K137Y) were ordered from GenScript Corporation (USA). The cDNAs contained NcoI restriction site at the 5″end and HindIII at the 3″end. The cDNAs were cloned as NcoI-HindIII fragments into bacterial expression vector pKKtac encoding the Ervinia carotovora's pectate lyase (pelB) signal sequence (Takkinen et al., Protein Eng. (4): 837-841, 1991) and expression plasmids were transformed into E. coli XL-1 Blue strain. The DNA sequences of the rBet v 1 and its mutants were verified by DNA sequencing (ABI 3100 Genetic Analyzer, Applied Biosystems), and are herein depicted as SEQ ID NO:s 1-3 and SEQ ID NO: 40 (SEQ ID NO: 1 wild type Bet v 1; SEQ ID NO: 2 Bet v 1 S99Y mutant; SEQ ID NO: 3 Bet v 1 K137Y mutant; SEQ ID NO: 40 Bet v 1 E101K mutant).

### Example 3

### Production of the Recombinant Bet v 1 Molecules

The rBet v 1 and its mutants were transformed into E. coli BL21 DE strain for bacterial expression. Single colonies were inoculated into 5 ml LB, 100 μg/ml ampicillin and 1% glucose and cultivated for 16 h at +37° C. with 220 rpm shaking. Cultivations were 1:50 diluted into 3×300 ml TB with 100 μg/ml ampicillin and cultivated at +37° C. until the OD600 reached 4. Protein expression was induced by the addition of IPTG to a final concentration of 1 mM and cells were cultivated for 16 h at RT with 170 rpm shaking. Cells were harvested by centrifugation for 15 min at 5000 g at +4° C. and the periplasmic fraction of the cells was isolated by an osmosis-shock method described by Boer et al., in Protein Expression & Purification, 2007(51): 216-226. The cell pellet equivalent of 900 ml culture was resuspended in 300 ml, 30 mM Tris/HCl, 20% sucrose, pH 8.0 and 1 mM EDTA, and incubated for 20 min under shaking on ice. The suspension was centrifuged for 20 min at 8000 g at 4° C. After this the pellet was resuspended in 75 ml of ice-cold 5 mM MgSO4 and shaken for 20 min at 4° C. on ice, and the osmotic shock fluid was harvested by centrifugation at 8000 g for 20 min at 4° C.

### Example 4

### Purification of the rBet v 1 Molecules

Periplasmic fractions were supplemented with 1 M NaCl and loaded onto a phenyl-Sepharose column (GE Healthcare) with 20 mM NaH₂PO₄, 1M NaCl, pH 5.0, the flow rate being 2 ml/min. Elution was performed as a linear gradient with 20 mM Tris-HCl, pH 9.3 supplemented with 7.5% isopropanol. Fractions containing recombinant Bet v 1 E101K and K137Y polypeptides were pooled, concentrated and subjected to Bio-Gel P60 size exclusion chromatography on a 200 ml column with a bed height of 460 mm and 1×PBS buffer flow rate of 0.3 ml/min. In the case of the rBet v 1 S99Y mutant an additional CM Sepharose™ Fast Flow (GE Healthcare) chromatography step was required before the size exclusion chromatography. The r Bet v 1 S99Y in 50 mM glycine pH 3.8 was subjected to the CM column and eluted with a linear NaCl gradient (5 mM-1M NaCl in 50 mM glycine pH 3.8).

Protein concentration of pooled rBet v 1 fractions was determined at 280 nm.

### Example 5

### Analysis of rBet v 1 and Mutants by Mass Spectrometry

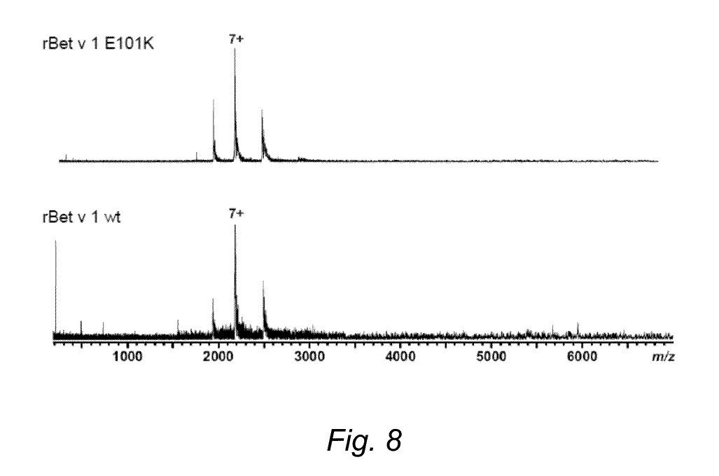

Mass-spectrometric experiments were performed with a 4.7 T Bruker BioAPEX-II ESI FT-ICR mass spectrometer (Bruker Daltonics, Billerica, Mass., USA) equipped with a conventional ESI source (Apollo-IITM). Native mass spectra: desalted allergen samples at concentration of 3 μM in 10 mM ammonium acetate buffer (pH 6.9) were directly infused at a flow rate of 1.5 mL/min with dry nitrogen serving as the drying (200 uC, 6 mbar) and nebulising gas. All instrumental parameters were optimized to maintain non-covalent interactions in the gas-phase and to maximize ion transmission at m/z 2000-3000. The same instrumental parameter settings were employed throughout to avoid any bias between different samples. Typically, 500-1000 co-added 128-kword time-domain transients were recorded and processed to 512-kword data prior to fast Fourier transform and magnitude calculation. Mass calibration was done externally with respect to the ions of an ES Tuning Mix (Agilent Technologies, Santa Clara, Calif., USA). Denaturated spectra were typically measured in acetonitrile/water/acetic acid solution. All data were acquired and processed with the use of Bruker XMASS 7.0.8 software. The native ESI FT-ICR mass spectra in FIG. 8 shows that the recombinant Bet v 1 mutant E101K folds similarly as the recombinant Bet v 1 wild type.

### Example 6

### Inhibition of Serum IgE Binding to Recombinant Bet v 1, Bet v 1 S99Y and E101K Polypeptides Analysed by a Competitive ELISA

The binding of a IgE serum sample of a birch pollen allergic person (E3) to biotinylated rBet v 1 immobilized on streptavidin wells was inhibited by increasing amounts of the rBet v 1 and rBet v 1 S99Y and E101K mutants. First, commercially available rBet v 1 (wild type, Biomay) was biotinylated using Sulfo-NHS-LC-biotin (Pierce) according to manufacturer's protocol. The biotinylated rBet v 1 (0.5 μg/well) was immobilized onto the streptavidin (SA) wells (Roche Diagnostics GmbH) followed by a washing step and the addition of E3 serum (1:6 dilution). After a 2-hour incubation at RT in a shaker and a washing step different amounts (4, 1, 0.25, 0.0625, 0.0156, and 0.0039 μg) of rBet v 1 were added and incubated for 2 h at RT in a shaker. After a washing step, the detection of bound IgE molecules was performed using a 1:1000 dilution of an AFOS-conjugated anti-human IgE antibody (Southern Biotech Associates Inc.) with an incubation for 1 h at RT in a shaker. Finally the substrate solution, p-nitrophenylphosphate (Sigma), was added and the absorbance values at 405 nm were measured (Varioscan, Thermo Electron Corporation).

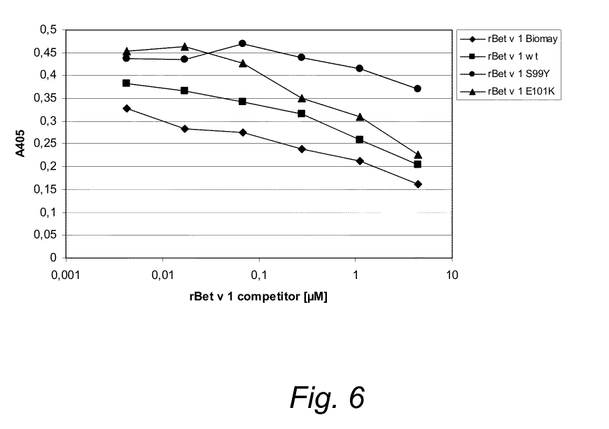

The result of serum IgE binding to r Bet v 1 polypeptides analysed by a competitive ELISA is shown in FIG. 6. The rBet v 1 proteins, wt, S99Y and E101K, were used for competing the E3 serum IgE binding to immobilized rBet v 1 (Biomay). Both rBet v 1 wild type molecules (a commercial one from Biomay and own produced) were inhibiting the IgE binding to the immobilised bet v 1. The Bet v 1 mutants, S99Y and E101K, showed reduced inhibition when compared to the rBet v 1 controls, especially in the lower concentration range of the inhibitor, indicating that these designed mutations are in the IgE epitope area of Bet v 1.

### Example 7

### Histamine Release Assay

The biological activity of the purified recombinant Bet v1 proteins (wt, S99Y and K137Y) in parallel with the commercial recombinant Bet v 1 (Biomay, Austria) was analysed by the method of passive sensitization of stripped basophiles and a subsequent challenge with the allergen molecules. The histamine release assay was done as an outsourced service at RefLab ApS, Copenhagen, Denmark, having an accredited histamine release assay method. The induction of the in vitro release of histamine from basophilic leukocytes by a commercial recombinant Bet v 1 (Biomay) and the three recombinant Bet v 1 proteins, wt, S99Y and K137Y was measured. Each of the four allergens was tested in the passive transfer test as a dose response study with the concentration range of: 20-0.06 ng/. Each concentration was tested in duplicate.

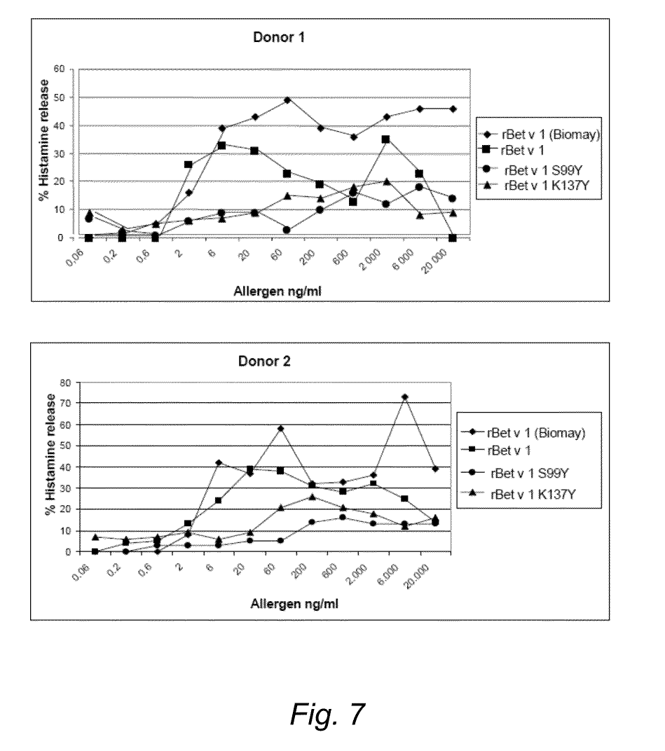

The assay was performed on two donors and the results are shown in FIG. 7 (donor 1 upper panel) and donor 2 (lower panel) with the serum of a Bet v 1 allergic person and purified allergens in a concentration range of 0.06-20 000 ng/ml (x-axis) the mean value of in duplicate measurements is shown. The percentage of histamine released into the supernatant is shown on the y-axis.

The three Bet V1 analogues (protein 2, 3 & 4) varied, showing that the rBet v 1 was equal to the reference extract, whereas rBet v 1 K137Y and rBet v 1 S99Y were 20 and 100 times, respectively, less biological active compared to the reference commercial recombinant Bet v 1 (Biomay).

It will be obvious to a person skilled in the art that, as the technology advances, the inventive concept can be implemented in various ways. The invention and its embodiments are not limited to the examples described above but may vary within the scope of the claims.

### Example 8

### Skin Prick Test

Skin prick tests (SPTs) with three voluntaries with two diagnosed birch pollen allergy and with one non-atopic person were performed with recombinant Bet v 1 polypeptides and relevant controls after the approval of the ethical committee of Helsinki University Central Hospital. The endotoxins of the recombinant Bet v 1 polypeptide preparations were removed by the Detoxi-Gel Endotoxin Removing Gel (Thermo: Cat. No. 20344) where after endotoxin content was analysed by ToxinSensor Endotoxin Detection System (GenScript: Cat. No. L00350C). The recombinant Bet v 1 polypeptide preparations were filter sterilised by Costar SPIN-X (Cat. No. 8160) and stored in aliquots at −20° C.

SPT was carried out using recombinant Bet v 1 a wt (Biomay) and E101K mutant at the concentrations of 50 and 5 μg/ml and a commercial birch pollen extract (AlkAbello). Sodium chloride (0.9%) and histamine dihydrochloride (AlkAbello) served as negative and positive controls, respectively. Before pricking the skin, lancets were set in the tubes containing the skin prick reagents. The responses were measured after 15 minutes and after 6 and 20 hours. The diameter of the skin response for histamine dihydrochloride in each tested individual was 5 mm after 15 min and this value was selected as positive (+) response (Table 2). The immediate skin reactions induced by the Bet v 1 mutant E101K with the concentration of 50 μg/ml or 5 μg/ml were comparable. Remarkably in the case of both allergic patients the skin reactions induced by the Bet v 1 E101K mutant disappeared within a notably shorter time than compared to the Bet v 1 wt.

TABLE 2
Results of the skin prick test

| | 15 min | 6 h | 20 h |
|---|---|---|---|
| Patient 1 | | | |
| Histamine dihydrochloride (10 mg/ml) | + | + | − |
| Birch extract 10HEP(AlkAbello) | + | − | − |
| rBet v 1 wt (50 μg/ml) | ++ | ++ | + |
| rBet v 1 wt (5 μg/ml) | + | + | − |
| rBet v 1 E101K (50 μg/ml) | ++ | − | − |
| rBet v 1 E101K (5 μg/ml) | + | − | − |
| Patient 2 | | | |
| Histamine dihydrochloride (10 mg/ml) | + | + | − |
| Birch extract 10HEP(AlkAbello) | + | + | − |
| rBet v 1 wt (50 μg/ml) | +++ | +++ | ++ |
| rBet v 1 wt (5 μg/ml) | + | + | + |
| rBet v 1 E101K (50 μg/ml) | +++ | − | − |
| rBet v 1 E101K (5 μg/ml) | ++ | − | − |
| Non-atopic person | | | |
| Histamine dihydrochloride (10 mg/ml) | + | − | − |
| Birch extract 10HEP(AlkAbello) | − | − | − |
| rBet v 1 wt (50 μg/ml) | − | − | − |
| rBet v 1 wt (5 μg/ml) | − | − | − |
| rBet v 1 E101K (50 μg/ml) | − | − | − |
| rBet v 1 E101K (5 μg/ml) | − | − | − |

+ diameter of the skin response ≧5 mm
++ diameter of the skin response ≧8 mm
+++ diameter of the skin response ≧11 mm

1. A recombinant birch pollen Bet v 1 polypeptide based on a wild type amino acid sequence as depicted in SEQ ID NO: 4 or any other Bet v1 wild type isoform, said polypeptide comprising at least one amino acid substitution at a position selected from the group consisting of amino acid residues 101, 137, 99, 80, 82, 84, 117, 119, 7, 9, 133, 141, and 145.

2. The polypeptide according to claim 1, wherein said amino acid sequence is selected from the group consisting of SEQ ID NO: 4-39.

3. The polypeptide according to claim 1 or 2, wherein the polypeptide comprises an amino acid sequence selected from the group consisting of SEQ ID NO: 41-53, or isoform thereof.

4. The polypeptide according claim 1, wherein the polypeptide comprises an amino acid sequence selected from the group consisting of
SEQ ID NO: 41, wherein amino acid 101 is K,
SEQ ID NO: 42, wherein amino acid 137 is K,
SEQ ID NO: 43, wherein amino acid 99 is K,
SEQ ID NO: 44, wherein amino acid 80 is Y,
SEQ ID NO: 45, wherein amino acid 82 is K,
SEQ ID NO: 46, wherein amino acid 84 is K,
SEQ ID NO: 47, wherein amino acid 117 is K,
SEQ ID NO: 48, wherein amino acid 119 is E,
SEQ ID NO: 49, wherein amino acid 7 is E,
SEQ ID NO: 50, wherein amino acid 9 is E,
SEQ ID NO: 51, wherein amino acid 133 is Y,
SEQ ID NO: 52, wherein amino acid 141 is K, and
SEQ ID NO: 53, wherein amino acid 145 is Y, or isoform thereof.

5. The polypeptide according to claim 1, wherein said amino acid substitution is located at amino acid position 101, 137 or 99.

6. The polypeptide according to claim 5, wherein S99 is replaced by tyrosine.

7. The polypeptide according to claim 5, wherein K137 is replaced by tyrosine.

8. The polypeptide according to claim 5, wherein E101 is replaced by lysine.

9. The polypeptide according to claim 1, having a histamine release capacity which is at least 20× reduced when compared to the histamine release capacity of the wild type Bet v1.

10. The polypeptide according to claim 9, wherein reduction in histamine release capacity is at least 100×.

11. The polypeptide according to claim 1, wherein there are altogether at least two, three or four amino acid substitutions.

12. A vaccine comprising at least one hypoallergenic polypeptide according to claim 1.

13. The vaccine according to claim 12 characterized in that said vaccine is for sublingual administration.

14. Use of a recombinant birch pollen Bet v1 polypeptide based on a wild type amino acid sequence as depicted in SEQ ID NO: 4 or any Bet v1 wild type variant thereof, said polypeptide comprising at least one amino acid substitution at a position selected from the group consisting of amino acid residues E101, K137, S99, K80, N82, S84, 5117, K119, T7, T9, V133, E141, and R145 as a vaccine.

15. A method of vaccinating against birch pollen allergy, said method comprising administering to a subject in need thereof a composition comprising at least one hypoallergenic polypeptide according to claim 1 and at least one pharmaceutically acceptable adjuvant.

16. A recombinant birch pollen Bet v 1 polypeptide based on a wild type amino acid sequence as depicted in SEQ ID NO: 4 or any other Bet v 1 wild type isoform, said polypeptide comprising at least one amino acid substitution at a position selected from the group consisting of amino acid residues 101, 137, 99, 80, 82, 84, 117, 119, 7, 9, 133, 141, and 145 for use as a vaccine.


SEQUENCE LISTING (53 sequences)

SEQ ID NO: 1 - DNA, 480 bp, Betula verrucosa
ggcgtgtttaactatgaaaccgaaaccaccagcgtgattccggcggcgcgtctgtttaaagcgtttattctggatggcgataacctgtttccgaaagtggcgccgcaggcgattagcagcgtggaaaacattgaaggcaacggcggcccgggcaccattaagaaaatcagcttcccggaaggctttccgttcaaatacgtgaaagatcgtgtggatgaagtggatcataccaacttcaaatacaactacagcgtgattgaaggcggcccgattggcgataccctggaaaaaattagcaacgaaattaaaattgtggcgaccccggatggcggcagcattctgaaaattagcaacaaatatcataccaaaggcgatcatgaagtgaaagcggaacaggtgaaagcgagcaaagaaatgggcgaaaccctgctgcgtgcggtggaaagctatctgctggcgcatagcgatgcgtataactaa

SEQ ID NO: 2 - DNA, 480 bp, Betula verrucosa
ggcgtgtttaactatgaaaccgaaaccaccagcgtgattccggcggcgcgtctgtttaaagcgtttattctggatggcgataacctgtttccgaaagtggcgccgcaggcgattagcagcgtggaaaacattgaaggcaacggcggcccgggcaccattaagaaaatcagcttcccggaaggctttccgttcaaatacgtgaaagatcgtgtggatgaagtggatcataccaacttcaaatacaactacagcgtgattgaaggcggcccgattggcgataccctggaaaaaatttataacgaaattaaaattgtggcgaccccggatggcggcagcattctgaaaattagcaacaaatatcataccaaaggcgatcatgaagtgaaagcggaacaggtgaaagcgagcaaagaaatgggcgaaaccctgctgcgtgcggtggaaagctatctgctggcgcatagcgatgcgtataactaa

SEQ ID NO: 3 - DNA, 480 bp, Betula verrucosa
ggcgtgtttaactatgaaaccgaaaccaccagcgtgattccggcggcgcgtctgtttaaagcgtttattctggatggcgataacctgtttccgaaagtggcgccgcaggcgattagcagcgtggaaaacattgaaggcaacggcggcccgggcaccattaagaaaatcagcttcccggaaggctttccgttcaaatacgtgaaagatcgtgtggatgaagtggatcataccaacttcaaatacaactacagcgtgattgaaggcggcccgattggcgataccctggaaaaaattagcaacgaaattaaaattgtggcgaccccggatggcggcagcattctgaaaattagcaacaaatatcataccaaaggcgatcatgaagtgaaagcggaacaggtgaaagcgagctatgaaatgggcgaaaccctgctgcgtgcggtggaaagctatctgctggcgcatagcgatgcgtataactaa

SEQ ID NO: 4 - protein, 159 aa, Betula verrucosa
  1epitope (2)..(6)
  2epitope (3)..(12)
  1epitope (70)..(75)
  1epitope (78)..(84)
  1epitope (96)..(103)
  2epitope (107)..(109)
  1epitope (115)..(121)
  2epitope (130)..(152)
GVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 5 - protein, 159 aa, Betula verrucosa; identical to SEQ ID NO: 4

SEQ ID NO: 6 - protein, 159 aa, Betula verrucosa; identical to SEQ ID NO: 4

SEQ ID NO: 7 - protein, 159 aa, Betula verrucosa
GVFNYETETTSVIPAARLFKAFILDGDNLFPKVAPQAISSVENIEGNGGPGTIKKISFPEGFPFKYVKDRVDEVDHTNFKYNYSVIEGGPVGDTLEKISNEIKIVATPDGGSILKISNKYHTKGDHEVKAEQVKASKEMGETLLRAVESYLLAHSDAYN

SEQ ID NO: 8 - protein, 159 aa, Betula verrucosa
GVFNYETEATSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 9 - protein, 159 aa, Betula verrucosa; identical to SEQ ID NO: 8

SEQ ID NO: 10 - protein, 159 aa, Betula verrucosa
GVFNYETETTSVIPAARLFKAFILDGDNLFPKVAPQAISSVENIEGNGGPGTIKKISFPEGLPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 11 - protein, 159 aa, Betula verrucosa
GVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMRETLLRAVESYLLAHSDAYN

SEQ ID NO: 12 - protein, 159 aa, Betula verrucosa
GVFNYETETTSVIPAARLFKAFILDGDNLVPKVAPQAISSVENIEGNGGPGTIKKISFPEGFPFKYVKDRVDEVDHTNFKYNYSVIEGGPMGDTLEKISNEIKIVATPDGGSILKISNKYHTKGDHEVKAEQVKASKEMGETLLRAVESYLLAHSDAYN

SEQ ID NO: 13 - protein, 159 aa, Betula verrucosa
GVFNYETETTSVIPAARLFKAFFLDGDNLFPKVAPQAISSVENIEGNGGPGTIKKISFPEGFPFRYVKDRVDEVDHTNFKYSYSVIEGGPVGDTLEKISNEIKIVATPDGGSILKISNKYHTKGDHEVKEEQIKASKEMGETLLRAVESYLLAHSDAYN

SEQ ID NO: 14 - protein, 159 aa, Betula verrucosa
GVFNYETEATSVIPAARLFKAFILDGDNLFPKVAPQAISSVENIEGNGGPGTIKKISFPEGIPFKYVKGRVDEVDHTNFKYSYSVIEGGPVGDTLEKISNEIKIVATPNGGSILKINNKYHTKGDHEVKAEQIKASKEMGETLLRAVESYLLAHSDAYN

SEQ ID NO: 15 - protein, 159 aa, Betula verrucosa
GVFNYEIEATSVIPAARLFKAFILDGDNLFPKVAPQAISSVENIEGNGGPGTIKKISFPEGFPFKYVKDRVDEVDHTNFKYSYSVIEGGPVGDTLEKISNEIKIVATPNGGSILKINNKYHTKGDHEVKAEQIKASKEMGETLLRAVESYLLAHSDAYN

SEQ ID NO: 16 - protein, 159 aa, Betula verrucosa; identical to SEQ ID NO: 15

SEQ ID NO: 17 - protein, 159 aa, Betula verrucosa
GVFNYETEATSVIPAARLFKAFILDGDNLFPKVAPQAISSVENIEGNGGPGTIKKISFPEGFPFKYVKDRVDEVDHTNFKYSYSVIEGGPVGDTLEKISNEIKIVATPNGGSILKINNKYHTKGDHEVKAEQIKASKEMGETLLRAVESYLLAHSDAYN

SEQ ID NO: 18 - protein, 159 aa, Betula verrucosa
GVFNYETEATSVIPAARLFKAFILDGDNLFPKVAPQAISSVENIEGNGGPGTIKKISFPEGIPFKYVKDRVDEVDHANFKYSYSVIEGGPVGDTLEKISNEIKIVATPDGGSILKISNKYHTKGDHEVKAEQIKASKEMGETLLRAVERYLLAHSDAYN

SEQ ID NO: 19 - protein, 159 aa, Betula verrucosa
GVFNYEIETTSVIPAARLFKAFILDGDNLVPKVAPQAISSVENIEGNGGPGTIKKINFPEGFPFKYVKDRVDEVDHTNFKYNYSVIEGGPVGDTLEKISNEIKIVATPDGGCVLKISNKYHTKGNHEVKAEQVKASKEMGETLLRAVESYLLAHSDAYN

SEQ ID NO: 20 - protein, 159 aa, Betula verrucosa; identical to SEQ ID NO: 19

SEQ ID NO: 21 - protein, 159 aa, Betula verrucosa
GVFNYESETTSVIPAARLFKAFILEGDNLIPKVAPQAISSVENIEGNGGPGTIKKINFPEGFPFKYVKDRVDEVDHTNFKYNYSVIEGGPVGDTLEKISNEIKIVATPDGGCVLKISNKYHTKGNHEVKAEQVKASKEMGETLLRAVESYLLAHSDAYN

SEQ ID NO: 22 - protein, 159 aa, Betula verrucosa
GVFNYETEATSVIPAARMFKAFILDGDKLVPKVAPQAISSVENIEGNGGPGTIKKINFPEGFPFKYVKDRVDEVDHTNFKYNYSVIEGGPVGDTLEKISNEIKIVATPDGGCVLKISNKYHTKGNHEVKAEQVKASKEMGETLLRAVESYLLAHSDAYN

SEQ ID NO: 23 - protein, 159 aa, Betula verrucosa
GVFNYETETTSVIPAARLFKAFILDGDNLFPKVAPQAISSVENIEGNGGPGTIKKISFPEGFPFKYVKDRVDEVDHTNFKYNYSVIEGGPVGDTLEKISNEIKIVATPDGGCVLKISNKYHTKGNHEVKAEQVKASKEMGETLLRAVESYLLAHSDAYN

SEQ ID NO: 24 - protein, 159 aa, Betula verrucosa
GVFNYETETTSVIPAARLFKAFILDGDNLFPKVAPQAISSVENIEGNGGPGTIKKISFPEGFPFKYVKDRVDEVDHTNFKYNYSVIEGGPIGDILEKISNEIKIVATPDGGCVLKISNKYHTKGNHEVKAEQVKASKEMGETLLRAVESYLLAHSDAYN

SEQ ID NO: 25 - protein, 159 aa, Betula verrucosa
GVFNYETETTSVIPAARLFKAFILDGDNLFPKVAPQAISSVENIEGNGGPGTIKKISFPGGLPFKYVKDRVDEVDHTNFKYNYSVIEGGPIGDTLEKISNEIKIVATPDGGCVLKISNKYHTKGNHEVKAEQVKASKEMGETLLRAVESYLLAHSDAYN

SEQ ID NO: 26 - protein, 159 aa, Betula verrucosa
GVFNYETETTSVIPAARLFKAFILDGDNLFPKVAPQAISSVENIEGNGGPGTIKKISFPEGFPFKYVKDRVDEVAHKNFKYSYSVIEGGPIGDTLEKISNEIKIVATPDGRSILKISNKYHTKGDHEVKAEQIKASKEMGETLLRAVESYLLAHSDAYN

SEQ ID NO: 27 - protein, 159 aa, Betula verrucosa
GVFNYETETTSVIPAARLFKAFILEGDTLIPKVAPQAISSVENIEGNGGPGTIKKITFPEGSPFKYVKERVDEVDHANFKYSYSMIEGGALGDTLEKICNEIKIVATPDGGSILKISNKYHTKGDHEMKAEHMKAIKEKGEALLRAVESYLLAHSDAYN

SEQ ID NO: 28 - protein, 159 aa, Betula verrucosa
GVFNYESETTSVIPAARLFKAFILEGDTLIPKVAPQAISSVENIEGNGGPGTIKKITFPEGSPFKYVKERVDEVDHANFKYSYSMIEGGALGDTLEKICNEIKIVATPDGGSILKISNKYHTKGDHEMKAEHMKAIKEKGEALLRAVESYLLAHSDAYN

SEQ ID NO: 29 - protein, 159 aa, Betula verrucosa
GVFNYESETTSVIPAARLFKAFILEGDTLIPKVAPQAISSVENIEGNGGPGTIKKITFPEGSPFKYVKERVDEVDHANFKYSYSMIEGGALGDTLEKICNEIKIVATPDGGSILKISNKYHTKGDQEMKAEHMKAIKEKGEALLRAVESYLLAHSDAYN

SEQ ID NO: 30 - protein, 159 aa, Betula verrucosa
GVFNYESETTSVIPAARLFKAFILDGDNLIPKVAPQAISSVENIEGNGGPGTIKKITFPEGSPFKYVKERVDEVDHANFKYSYSMIEGGALGDTLEKICNEIKIVATPDGGSILKISNKYHTKGDHEMKAEHMKAIKEKGEALLRAVESYLLAHSDAYN

SEQ ID NO: 31 - protein, 159 aa, Betula verrucosa; identical to SEQ ID NO: 30

SEQ ID NO: 32 - protein, 159 aa, Betula verrucosa
GVFNYETETTSVIPAARLFKAFILDGDNLIPKVAPQAISSVENIEGNGGPGTIKKITFPEGSPFKYVKERVDEVDHANFKYAYSMIEGGALGDTLEKICNEIKIVATPDGGSILKISNKYHTKGDHEMKAEHMKAIKEKGEALLRAVESYLLAHSDAYN

SEQ ID NO: 33 - protein, 159 aa, Betula verrucosa
GVFNYETETTSVIPAARLFKAFILDGDNLIPKVAPQAISSVENIEGNGGPGTIKKITFPEGSPFKYVKERVDEVDHANFKYSYSMIEGGALGDTLEKICNEIKIVATPDGGSILKISNKYHTKGDHEMKAEHMKAIKEKGEALLRAVESYLLAHSDAYN

SEQ ID NO: 34 - protein, 159 aa, Betula verrucosa
GVFNYETETTSVIPAARLFKAFILDGDNLIPKVAPQAISSVENIEGNGGPGTIKKITFPEGSPFKYVKERVDEVDHANFKYSYSMIEGGALGDTLEKICNEIKLVATPDGGSILKISNKYHTKGDHEMKAEHMKAIKEKAEALLRAVESYLLAHSDAYN

SEQ ID NO: 35 - protein, 159 aa, Betula verrucosa
GVFDYEGETTSVIPAARLFKAFILDGDNLIPKVAPQTVSCVENIEGNGGPGTIKKITFPEGSPFKYVKERVDEVDHVNFKYSYSVIEGGAVGDTLEKICNEIKIVPAPGGGSILKISNKYHTKGNHEMKAEQIKASKEKAEALFRAVESYLLAHSDAYN

SEQ ID NO: 36 - protein, 159 aa, Betula verrucosa
GVFDYEGETTSVIPAARLFKAFILDGDNLIPKVAPQAVSCVENIEGNGGPGTIKKITFPEGSPFKYVKERVDEVDRVNFKYSYSVIEGGAVGDTLEKICNEIKIVPAPGGGSILKISNKYHTKGNHEMKAEQIKASKEKAEALFRAVESYLLAHSDAYN

SEQ ID NO: 37 - protein, 159 aa, Betula verrucosa
GVFNYETETPSVIPAARLFKAFILDGDKLLPKVAPEAVSSVENIEGNGGPGTIKKITFPEGSPFKYVKERVDEVDRVNFKYSFSVIEGGAVGDALEKVCNEIKIVAAPDGGSILKISNKFHTKGDHEINAEQIKIEKEKAEGLLKAVESYHLAHSDAYN

SEQ ID NO: 38 - protein, 159 aa, Betula verrucosa
GVFNYETEATSVIPAARLFKASILDGDNLFPKVAPQAISSVENIEGNGGPGTIKKISFPEGSPFKYVKERVDEVDRVNFKYSFSVIEGGAVGDALEKVCNEIKIVAAPDGGSILKISNKFHTKGDHEINAEQIKIEKEKAVGLLKAVESYLLAHSDAYN

SEQ ID NO: 39 - protein, 159 aa, Betula verrucosa
GVFNYEDEATSVIAPARLFKSFVLDADNLIPKVAPENVSSAENIEGNGGPGTIKKITFPEGSHFKYMKHRVDEIDHANFKYCYSIIEGGPLGDTLEKISYEIKIVAAPGGGSILKITSKYHTKGDISLNEEEIKAGKEKGAGLFKAVENYLVAHPNAYN

SEQ ID NO: 40 - DNA, 480 bp, Betula verrucosa
ggcgtgtttaactatgaaaccgaaaccaccagcgtgattccggcggcgcgtctgtttaaagcgtttattctggatggcgataacctgtttccgaaagtggcgccgcaggcgattagcagcgtggaaaacattgaaggcaacggcggcccgggcaccattaagaaaatcagcttcccggaaggctttccgttcaaatacgtgaaagatcgtgtggatgaagtggatcataccaacttcaaatacaactacagcgtgattgaaggcggcccgattggcgataccctggaaaaaattagcaacaaaattaaaattgtggcgaccccggatggcggcagcattctgaaaattagcaacaaatatcataccaaaggcgatcatgaagtgaaagcggaacaggtgaaagcgagcaaagaaatgggcgaaaccctgctgcgtgcggtggaaagctatctgctggcgcatagcgatgcgtataactaa

SEQ ID NO: 41 - protein, 159 aa, Betula verrucosa
  MISC_FEATURE (7)..(7): X is T, E, Y, K, R, or W
GVFNYEXEXTSVIPAARLFKAFILDGDNLFPKVAPQAISSVENIEGNGGPGTIKKISFPEGFPFKYVKDRVDEVDHTNFXYXYXVIEGGPIGDTLEKIXNXIKIVATPDGGSILKIXNXYHTKGDHEVKAEQXKASXEMGXTLLXAVESYLLAHSDAYN

SEQ ID NO: 42 - protein, 159 aa, Betula verrucosa; identical to SEQ ID NO: 41
  MISC_FEATURE (7)..(7): X is T, E, Y, K, R, or W

SEQ ID NO: 43 - protein, 159 aa, Betula verrucosa; identical to SEQ ID NO: 41
  MISC_FEATURE (7)..(7): X is T, E, Y, K, R, or W

SEQ ID NO: 44 - protein, 159 aa, Betula verrucosa; identical to SEQ ID NO: 41
  MISC_FEATURE (7)..(7): X is T, E, Y, K, R, or W

SEQ ID NO: 45 - protein, 159 aa, Betula verrucosa; identical to SEQ ID NO: 41
  MISC_FEATURE (7)..(7): X is T, E, Y, K, R, or W

SEQ ID NO: 46 - protein, 159 aa, Betula verrucosa; identical to SEQ ID NO: 41
  MISC_FEATURE (7)..(7): X is T, E, Y, K, R, or W

SEQ ID NO: 47 - protein, 159 aa, Betula verrucosa; identical to SEQ ID NO: 41
  MISC_FEATURE (7)..(7): X is T, E, Y, K, R, or W

SEQ ID NO: 48 - protein, 159 aa, Betula verrucosa; identical to SEQ ID NO: 41
  MISC_FEATURE (7)..(7): X is T, E, Y, K, R, or W

SEQ ID NO: 49 - protein, 159 aa, Betula verrucosa; identical to SEQ ID NO: 41
  MISC_FEATURE (7)..(7): X is E, Y, K, R, or W

SEQ ID NO: 50 - protein, 159 aa, Betula verrucosa; identical to SEQ ID NO: 41
  MISC_FEATURE (7)..(7): X is T, E, Y, K, R, or W

SEQ ID NO: 51 - protein, 159 aa, Betula verrucosa; identical to SEQ ID NO: 41
  MISC_FEATURE (7)..(7): X is T, E, Y, K, R, or W

SEQ ID NO: 52 - protein, 159 aa, Betula verrucosa; identical to SEQ ID NO: 41
  MISC_FEATURE (7)..(7): X is T, E, Y, K, R, or W

SEQ ID NO: 53 - protein, 159 aa, Betula verrucosa; identical to SEQ ID NO: 41
  MISC_FEATURE (7)..(7): X is T, E, Y, K, R, or W